(19) United States
(12) Patent Application Publication
(10) Pub. No.: US 2025/0327654 A1
(43) Pub. Date: Oct. 23, 2025

(54) SELF-MIXING INTERFEROMETRY SENSOR MODULE FOR MULTILAYER TARGET DETECTION, ELECTRONIC DEVICE AND METHOD OF MULTILAYER TARGET DETECTION

(71) Applicant: ams International AG, Jona (CH)

(72) Inventors: Daniel NAJER, Rotkreuz (CH); Christian RABUS, München (DE); Tanja KOCH, Bruckmühl (DE); Ferran SUAREZ, Rüschlikon (CH)

(21) Appl. No.: 18/869,954

(22) PCT Filed: May 16, 2023
(86) PCT No.: PCT/EP2023/063111

§ 371 Date: Nov. 27, 2024

(30) Foreign Application Priority Data
May 30, 2022 (DE) 10 2022 113 557.8

(51) Int. Cl. G01B 9/02; H01S 5/00; H01S 5/183

(52) U.S. Cl. CPC G01B 9/02092; H01S 5/0028; H01S 5/183; G01B 2290/45; G01B 2290/70

(57) ABSTRACT

A self-mixing interferometry sensor module for multilayer target detection includes a light emitter, a detector unit and an array of light detectors. The light emitter is operable to emit coherent electromagnetic radiation out of the sensor module, and undergo self-mixing interference (SMI) caused by reflections of the emitted electromagnetic radiation from layers of different depths of a multilayer target to be placed outside the sensor module. The detector unit is operable to generate an SMI output signal indicative of the SMI of the light emitter. Light detectors of the array are operable to generate auxiliary output signals indicative of a distribution of relative reflections of the emitted electromagnetic radiation from layers of different depths.

This disclosure relates to a self-mixing interferometry sensor module for multilayer target detection, an electronic device and a method of multilayer target detection.

Self-Mixing Interferometry, SMI for short, is an interferometric method to detect and investigate a target (e.g. with a single interface) in front of a laser source, such as a vertical cavity surface emitting laser (VCSEL). By making use of optical feedback and retro reflection from the target into the laser cavity, the intra-cavity field mixes with the reflected field. This leads to interference effects within the laser cavity, which are detectable via small changes in the emitted laser power or junction voltage across the laser diode. Changes in laser power can be probed anywhere around or behind the laser source by making use of scattered laser photons. These power or voltage changes are the result of interferometric effects or fringes that can appear due to a steady target (combined with laser wavelength modulation), a displaced target (combined with constant laser wavelength) or a moving target (Doppler shift of reflected photons from target).

A simple picture of an SMI sensor pointing at a target is that of a three-mirror model, where the laser itself comprises two mirrors and the external target acts as a third mirror. SMI at multilayer targets will change from a three-mirror model to an (N+2)-mirror model, where N is the number of target interfaces or layers. This considerably complicates the SMI signals; photons entering different layers of the target (i.e. travelling different path lengths) may contribute differently to the overall interference happening inside the laser. Typically, it remains unclear how many photons travel into what depth into the multilayer target.

Thus, an object to be achieved is to provide an SMI sensor module for electronic devices that overcomes the aforementioned limitations and provides multilayer target detection. A further object is to provide an electronic device comprising such a sensor module and a method of multilayer target detection.

These objectives are achieved with the subject-matter of the independent claims. Further developments and embodiments are described in dependent claims.

The following relates to an improved concept in the field of optical sensing. One aspect relates to the idea that self-mixing interferometry can be complemented with spatially offset photodetection to allow for an improved multilayer target detection. This aspect involves making use of the DC component of an SMI output signal to obtain an estimate of travel depth of photons depending on their detection offset with respect to the light source.

In at least one embodiment, a self-mixing interferometry sensor module for multilayer target detection comprises a light emitter, a detector unit and an array of light detectors. The array of light detectors comprises a number of light detectors.

In operation, the light emitter emits out of the sensor module coherent electromagnetic radiation. Furthermore, the light emitter undergoes self-mixing interference, SMI, which is caused by reflections of the emitted electromagnetic radiation from layers of different depths of a multilayer target to be placed outside the sensor module.

The detector unit generates an SMI output signal, which is indicative of the SMI of the light emitter. Furthermore, the light detectors of the array generate auxiliary output signals, which are indicative of a distribution of relative reflections of the emitted electromagnetic radiation from layers of different depths.

For example, the light emitter is placed in the sensor module to enable self-mixing interference, and typically comprises a cavity resonator, into which at least a fraction of the light emitted by the light emitter can be reflected, or back-scattered, from the multilayer target outside the module. For example, the light emitter is implemented as a semiconductor laser diode and comprises a laser cavity. The light emitter is configured to emit coherent light, e.g. in an infrared (IR), visible or ultraviolet (UV) range of the electromagnetic spectrum, out of the sensor module. The light emitter can be configured to generate continuous emission or to emit light in a pulsed fashion, the latter potentially aiding in achieving an overall reduction in power consumption.

Back-injection of the emitted light into the cavity is due to reflections from layers of different depths of the multilayer target outside the module. In fact, the light is reflected off different layers at defined depths or distances.

Consequently, the light emitter is subject to self-mixing interference caused by reflections of different depths.

When no target is present outside the module in the field of emission of the light emitter, i.e. no interception and reflection of light occurs, then no self-mixing interference occurs within the light emitter. However, when the emitted electromagnetic field from the laser cavity is reflected back into the cavity, it may change phase at the layers of the multilayer target, as the layers are at different target distances. This causes a modulation in the amplitude and/or frequency of the light emitter's electromagnetic light field due to interference. The self-mixing interference generates periodic fringes in the output signal of the light emitter, which is detected as the SMI output signal by the detector unit. More accurately, SMI modulates the optical power (e.g. observed by measuring it in a light detector, e.g. as photo-current) and the threshold gain (which can be detected by monitoring a laser voltage or laser current, for example). Another way of generating SMI is through modulation of an emission wavelength, e.g. ramping a laser current periodically (via triangular function current ramp or changing the laser cavity via a MEMS mirror).

As discussed above, SMI eventually alters a property of the light emitter. This property is indirectly measured by means of the detector unit, which generates the SMI output signal as a function of said property, or change of said property. The SMI output signal may be measured as current or voltage, for example. Thus, the detector unit may have means, e.g. active or passive circuitry, to measure said change as an electronic property.

The SMI output signal includes information of different depths and distances of the multilayer target. Typically, with a multilayer target photons entering different layers of the target travel different path lengths and, thus, contribute differently to the overall interference happening inside the light emitter. Without additional information, it usually remains unclear how many photons travel into what depth into the multilayer target. The resulting SMI picture changes from a three-mirror model, as discussed in the introduction, to an (N+2)-mirror model, where N is the number of target layers or interfaces. Furthermore, a multilayer target is a common case rather than an exception. For example, a human body part such as a finger has different skin in different depths.

The array of light detectors provides additional information in the form of the auxiliary output signals. For example, two pieces of information can be extracted from the array: photon travel depths into target (from a DC component of auxiliary signals) and the SMI output signal (from an AC component of signal).

The light detectors are spaced from the light emitter, and, thus, reflected light from the layers of the multilayer target contribute differently to output signals generated by the light detectors in the array. Thus, the auxiliary output signals allow to map a distribution of relative reflections of the emitted electromagnetic radiation from layers of different depths. This distribution allows to interpret the SMI output signal of the light emitter. This may provide a better understanding of a multilayer target by measuring which fraction of laser power enters which depth of the multilayer target. Ultimately, the distribution of relative reflections may allow to identify the contributions of the individual layers of the multilayer target to the overall SMI output signal. However, this may need to be supported by means of a reflection and scattering model of the multilayer target itself.

The proposed concept allows to determine what part of the emitted light travels to what depth of the multilayer target. This can be done via the spatially offset light detectors, where the radial distance of a light detector to the SMI light emitter is related to detection of photons reflected at a certain target depth. In other words, the detector furthest away from the laser emitter measures the photons that travel more deeply into the multilayer target.

The light detector array combined with SMI detection allows to resolve photon travel depth into a multilayer system, enabling an extensive investigation of multilayer targets. Robust sensing modality allows to investigate layered objects/multilayer targets such as human skin. The proposed concept may find applications in the consumer or medical field, including health monitoring systems, smartphones, wearables (smart watches, smart glasses and smart patches). Applications include non-invasive sensing (e.g. vital signs) of multilayer targets, such as human skin (vibrocardiography, blood flow sensing, blood pressure analysis . . . ), for example.

In at least one embodiment, the array of light detectors comprises a one-dimensional array or a two-dimensional array of light detectors. Neighboring light detectors, or photodetectors, are separated by a spatial offset.

The light detectors can be implemented by means of photodiodes, SPADs, or other types of semiconductor light detectors. For example, a row or column of the array is aligned with respect to the light emitter, so that light striking a light detector is reflected at different layers as one moves outwards along the row or column of the array.

Spatial offset determines a radial distance to the light emitter and, thus, relates to the detection of photons reflected at a certain target depth. In other words, the light detector furthest away from the laser source measures mostly, or exclusively, the photons that travel more deeply into the multilayer target. This allows to further understand a multilayer target by measuring which fraction of emitted light enters which depth of the multilayer target.

In at least one embodiment, the array of light detectors forms an image sensor. The light detectors are spatially offset by design of the image sensor. The image sensor may be implemented as a charge-coupled device (CCD) or a Complementary Metal-Oxide Semiconductor (CMOS) image sensor, for example. An image sensor allows to record the auxiliary output signals as an image and can be combined with Speckle imaging, for example.

In at least one embodiment, the light emitter comprises a semiconductor laser diode, resonant cavity light emitting device or vertical cavity surface emitting laser, VCSEL, diode. These devices feature coherent emission to generate SMI fringes. A resonant cavity light emitting device can be considered a semiconductor device which is operable to emit coherent light based on a resonance process. In this process, the resonant cavity light emitting device may directly convert electrical energy into light, e.g. when pumped directly with an electrical current to create amplified stimulated emission.

VCSELs are an example of a resonant-cavity light emitting device and feature a beam emission that is perpendicular to a main extension plane of a top surface of the VCSEL. The VCSEL diode can be formed from semiconductor layers on a substrate, wherein the semiconductor layers comprise two distributed

Bragg reflectors (DBRs) enclosing active region layers in between and thus forming a cavity. VCSELs and their principle of operation are a well-known concept and are not further detailed in this disclosure. For example, the VCSEL diode is configured to have an emission wavelength of 940 nm, 850 nm, or another wavelength. The VCSEL diode can be configured to emit coherent laser light when forward biased, for instance.

In at least one embodiment, the detector unit is operable to detect a junction voltage of the light emitter. In turn, the SMI output signal constitutes a function of said junction voltage. Junction voltage is one possible electronic property of the light emitter which may change as a result of SMI. For example, the detector unit comprises a voltage meter to detect the junction voltage.

In at least one embodiment, the detector unit is operable to detect an optical power output of the light emitter. In turn, the SMI output signal is generated as a function of said optical power output. Optical power is another possible property of the light emitters which may change as a result of SMI. For example, the detector unit comprises a light detector, such as a photodiode, or a photodiode array to detect optical power output.

In at least one embodiment, the module comprises further light emitters. The light emitters are operable to emit coherent electromagnetic radiation with a defined wavelength out of the sensor module. Each light emitter may undergo SMI, caused by reflections of the emitted electromagnetic radiation from layers of different depths of a multilayer target to be placed outside the sensor module. At least two light emitters are operable to emit coherent electromagnetic radiation with different defined wavelengths.

Light emitted by the light emitter may reach different layers of different depths of the multilayer target with different characteristics depending on wavelength. For example, a layer of the target may have a higher or lower absorption at a defined wavelength as compared to another wavelength. Furthermore, reflection or scattering at layers may also be depending on wavelength. Thus, further light emitters with different emission wavelength allow to include further information on how different layers of the target contribute to the overall SMI output signals. In addition, the light detectors may also be operable to spectrally resolve the detected auxiliary output signals, e.g. by means of dedicated filters.

In at least one embodiment, the array of light detectors, the detector unit and/or at least one light emitter form an integrated semiconductor device, such as a CMOS integrated circuit device, on a common substrate. In addition, or alternatively, the sensor module comprises a sensor package into which the array of light detectors, detector unit and, optionally, the light emitter(s) and/or further components such as an electronic processing unit, or the integrated semiconductor device formed by the array of light detectors, detector unit and/or at least one light emitter, are integrated.

In at least one embodiment, the module further comprises an electronic processing unit, which is operable to determine from the generated SMI output signal and auxiliary output signals a depth profile of the multilayer target. Thus, the depth profile can be determined and provided by an on-chip component and may not need additional processing outside the module.

In at least one embodiment, at least some of the light detectors are operable to generate the auxiliary output signals indicative of a distribution of relative reflections of the emitted electromagnetic radiation from layers of different depths as a function of polarization.

Unpolarized light emitted by the light emitter can be polarized by reflection at an angle from a dielectric surface. Polarized light emitted by the light emitter can be changed in its polarization due to reflection. The light detectors may be complemented with polarizers in order to determine a state of polarization. Depending on the nature of the multilayer target, a polarization state, or several states, may be attributed to the layers of the target.

In at least one embodiment, an optical element, such as a refractive, diffractive or meta-lens, is arranged in front of the light emitter. The optical element can be used to collimate or focus a diverging beam from the light emitter and/or provide polarization control, for instance. In addition, or alternatively, another optical element, such as a single microlens or microlens array, is arranged in front of the light detectors. This optical element can be used to increase the signal on the light detectors.

In at least one embodiment, an electronic device comprises a self-mixing interferometry sensor module according to one or more of the aforementioned aspects. Furthermore, the device comprises a housing, which further comprises the sensor module and a support surface. The multilayer target can be placed on the support surface. In this position, the housing is configured to position the light emitters at a distance from the multilayer target. As a consequence, the light emitter may essentially be perpendicular with respect to the support surface. The light detectors have a spatial offset with respect to a surface normal of the support surface and with respect to the light emitter.

In at least one embodiment, the module further comprises a processing unit, which is configured to determine, from an output of the module, a displacement or a movement of a sub-surface feature associated with at least one layer of the multilayer object. The output of the module may be the SMI and auxiliary output signals, or the SMI corrected in view of the auxiliary output signals, for example.

The processing unit can be a central processing unit, CPU, of the wearable electronic device, or a system-on-a-chip, SOC, that is dedicated to process output signals of the light emitters, for instance. The processing unit can be used instead, as the or as an addition to the electronic processing unit of the module.

For example, the processing unit interprets the SMI output signal in view of the auxiliary output signals and determines a displacement or a movement of a layer feature as a result of such interpretation. For example, the SMI output signal can be reduced to a signal component from a desired layer of the multilayer target.

In at least one embodiment, the processing unit is operable to receive as an output of the module at least one SMI output signal and the auxiliary output signals. The processing unit is operable to determine the displacement or movement of a sub-surface feature as a function of the SMI output signal and the auxiliary output signals.

In at least one embodiment, the processing unit is further operable to combine the output of the module with a Speckle image.

Speckle imaging originates from astronomical imaging and relates to high-resolution imaging based on the analysis of large numbers of short exposures that freeze the variation in the image. The image sensor may integrate a large number of exposures. The images are dependent on the different depths of the layers. Thus, if depth (or distance) changes, e.g. due to a changing parameter related to a given layer, this may be apparent in the images. These changes can be extracted by way of Speckle image processing and be related to the SMI output signal.

Further embodiments of the electronic device become apparent to the skilled reader from the aforementioned embodiments of the self-mixing interferometry sensor module, and vice-versa.

Furthermore, a method of detecting a multilayer object is provided, comprising at least the following steps.

One step includes placing a multilayer target outside a sensor module. Another step includes emitting coherent electromagnetic radiation out of the sensor module (10) by means of a light emitter. Another step includes generating self-mixing interference, SMI, in the light emitter caused by reflections of the emitted electromagnetic radiation from layers of different depths of the multilayer target to be placed outside the sensor module. Another step includes generating an SMI output signal indicative of the SMI of the light emitter. Another step includes using an array of light detectors, generating auxiliary output signals indicative of a distribution of relative reflections of the emitted electromagnetic radiation from layers of different depths.

Further embodiments of the method become apparent to the skilled reader from the aforementioned embodiments of the self-mixing interferometry sensor module and of the electronic device, and vice-versa.

The following description of figures may further illustrate and explain aspects of the self-mixing interferometry sensor module, electronic device and the method of multilayer target detection. Components and parts of the self-mixing interferometry sensor that are functionally identical or have an identical effect are denoted by identical reference symbols. Identical or effectively identical components and parts might be described only with respect to the figures where they occur first. Their description is not necessarily repeated in successive figures.

### IN THE FIGURES

FIG. 1 shows an exemplary embodiment of a self-mixing interferometry sensor module, and

FIG. 2 shows another exemplary embodiment of a self-mixing interferometry sensor module.

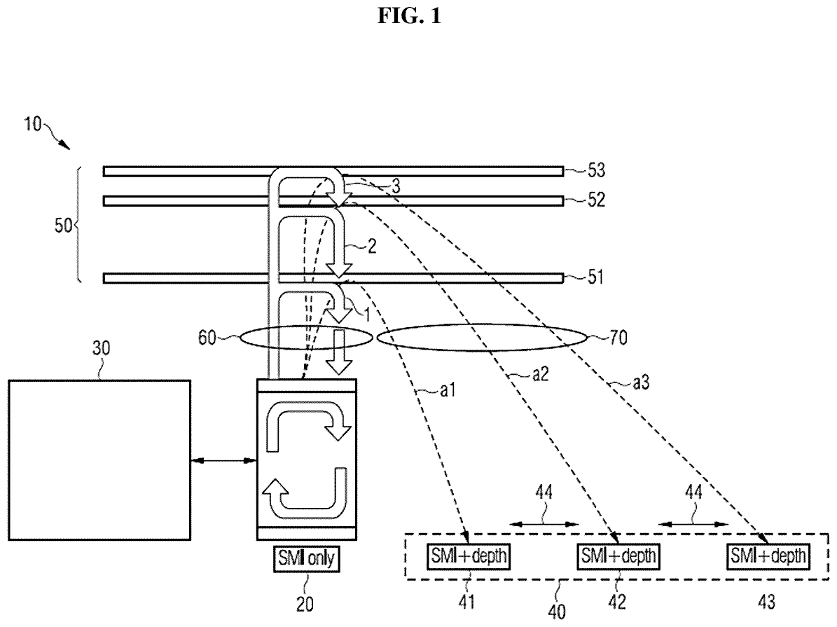

FIG. 1 shows an exemplary embodiment of a self-mixing interferometry sensor module. The self-mixing interferometry sensor module 10 comprises a light emitter 20, a detector unit 30 and an array of light detectors 40. Optionally, an optical element 60 (e.g. a refractive, diffractive or meta- lens) in front of the light emitter can be used to collimate or focus the diverging beam from the light emitter (or polarization control, for instance), and an optical element 70 (single microlens or microlens array) can be used to increase the signal on the light detectors.

The sensor module can be implemented as a sensor package and/or an integrated semiconductor device, into which the light emitter, detector unit and array of light detectors are integrated. For example, the detector unit and array of light detectors and, optionally, additional components such as an electronic processing unit (not shown) and/or a laser driver as a means to drive the light emitter 20 form an integrated semiconductor device, such as a CMOS integrated circuit device, on a common substrate. The light emitter can either be integrated into the integrated semiconductor device or be electrically connected to the integrated semiconductor device as an external component. The sensor module can be integrated into and electrically connected to an electronic device (not shown).

The light emitter 20 in this example is implemented as a vertical cavity surface emitting laser, or VCSEL, diode. A VCSEL is an example of a resonant cavity light emitting device. The VCSEL comprises semiconductor layers with distributed Bragg reflectors (not shown) which enclose active region layers in between and thus form a cavity. VCSELS feature a beam emission of coherent electromagnetic radiation that is perpendicular to a main extension plane of a top surface of the VCSEL. For example, the VCSEL diodes are configured to have an emission wavelength in the infrared range, e.g. at 940 nm or 850 nm. The light emitter (or VCSEL) serves as both illuminator and sensor as well as filter.

The detector unit 30 is shown as a schematic building block. The detector unit comprises means, e.g. active or passive circuitry, to measure an optical or electronic property of the light emitter 20. For example, the detector unit comprises a current or voltage meter to detect a junction voltage of the light emitter. Junction voltage is one possible electronic property of the light emitters and may change as a result of self-mixing interference. In addition or alternatively, the detector unit comprises a light detector, such as a photodiode, to detect an optical power output of the light emitter. The optical power output is a possible optical property of the light emitters and may change as a result of self-mixing interference. In some embodiments, the light detector can be epitaxially integrated with the light emitter 20 e.g. during the epitaxy of the light emitter 20.

Furthermore, the array of light detectors 40 comprises light detectors, such as individual photodiodes 41, 42, 43, which are arranged as an array with a spatial offset 44 between neighboring detectors. The array can be a one-dimensional array or a two-dimensional array, for example.

For example, the self-mixing interferometry sensor module is arranged in an electronic device, e.g. a health monitoring system, a smartphone, a wearable electronic device, such as a smart watch, smart glasses, smart patches, and the like, comprising the sensor module. Typically, the electronic device comprises a housing with an aperture, e.g. as part of a cover section. The sensor module is placed or mounted in the housing and positioned with respect to the aperture. A multilayer target 50, such as a finger or other human body part, can be placed on the aperture in order to conduct a measurement with the module.

The housing provides a defined distance between the multilayer target 50 and the sensor module 10. For example, the module is placed inside the housing such that the light emitter 20 essentially lies on or close to a common optical axis, which runs through the optional lens 60, the aperture and the light emitter. For example, the light emitter is placed perpendicular to a main surface of the housing comprising the aperture, e.g. a contact surface. This way, the light emitter can be placed perpendicular to a surface or layer 51 of the target 50, e.g. a skin surface. Light entering the housing via the aperture can reach the light emitter. Due to the spatial offset 44 the array of light detectors 40 is offset with respect to the common optical axis of the light emitter 20. However, light entering the housing via the aperture can reach the light detectors at an angle with respect to the main surface of the housing, which is indicated as arrows a1, a2, and a3, respectively. These originate from reflection or scattering events 1, 2, 3 at a first layer 51, a second layer 52 and a third layer 53, respectively. An optional lens 70 can be arranged in front of the light detectors to increase the signal-to-noise ratio.

Operation of the sensor module makes use of the DC component of the SMI output signal to obtain the travelling depth of photons depending on their detection offset with respect to the light emitter 20. While a number of spatially distributed light detectors 41, 42, 43 measure photons preferably from a certain target (in their DC component), each light detector from the array additionally carries information (in its AC component) from the interferences inside the laser (i.e. SMI signal). This way, the photodetector array can be used to both detect photon penetration depths as well as SMI parameters from the target (e.g. distance, displacement or speed).

The drawing illustrates a possible measurement. The light emitter 20 operates based on a laser process 4 (indicated by the circular arrows in the drawing). As a result, the light emitter emits coherent electromagnetic radiation out of the sensor module 10 towards a multilayer target 50, which is placed in a field-of-view of the emitter. Light may be reflected or backscattered (see events 1, 2, 3 in the drawing) at layers 51, 52, 53 of different depths of the multilayer target 50. Fractions of reflected or backscattered light eventually are coupled back into the light emitter, which then undergoes self-mixing interference. Further fractions of the reflected or backscattered light, however, do not travel back to the light emitter but rather strike the array of light detectors 20 instead. These fractions relate to the reflections at the layers at different angles as indicated by the arrows a1, a2, a3, respectively.

The detector unit 30 generates an SMI output signal, which is indicative of the SMI of the light emitter 20. The SMI output signal depends on the fractions of reinjected light, which have been reflected or back-scattered off the layers 51, 52, 53 of the multilayer target 50. In fact, due to SMI the SMI output signal has an AC component, which can be related to absolute or relative distance or speed of features of the individual layers. The SMI output signal may also have a DC component, which typically carries no useful information.

The light detectors 41, 42, 43 receive the fractions which have been reflected or back-scattered off the layers 51, 52, 53 of the multilayer target 50 but at an angle. These fractions give rise to respective auxiliary output signals of the light detectors 41, 42, 43. Together these auxiliary output signals are indicative of a distribution of relative reflections of the emitted electromagnetic radiation from layers of different depths.

The overall SMI output signal can be considered a superposition of partial SMI signals due to reflections off the layers 51, 52, 53 of the multilayer target 50. The light detectors are spaced from the light emitter and, thus, reflected light from the layers 51, 52, 53 of the multilayer target contribute differently to the auxiliary output signals. Thus, the auxiliary output signals allow to map a distribution of relative reflections of the emitted electromagnetic radiation from layers 51, 52, 53 of different depths. This distribution allows to interpret the SMI output signal of the light emitter 20. This may provide a better understanding of a multilayer target by measuring which fraction of laser power enters which depth of the multilayer target. Ultimately, the distribution of relative reflections may allow to identify the contributions of the individual layers of the multilayer target to the overall SMI output signal. However, this may need to be supported by means of a reflection and scattering model of the multilayer target itself.

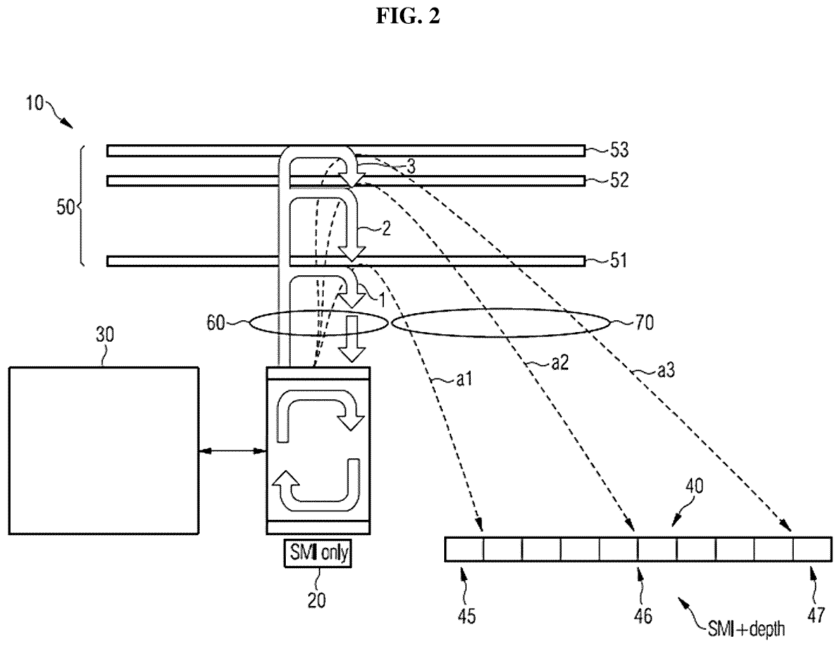

FIG. 2 shows another exemplary embodiment of a self-mixing interferometry sensor module. This setup differs from the previous one in FIG. 1 in that the light detector array 40 is implemented as an image sensor with pixels (see representative pixels 45, 46, 47 in the drawing). This way, the depth resolution increases and potential speckle patterns from the multilayer target can be measured as in Speckle Imaging. Speckle Imaging can be used to implement high-resolution imaging, e.g. to detect blood oxygenation, while the SMI detection by means of the light emitter could be used to detect motion of the target surface or within the target, including the flow of blood through vessels.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment.

Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

This patent application claims the priority of German patent application 102022113557.8, the disclosure content of which is hereby incorporated by reference.

### References

a1 arrow
a2 arrow
a3 arrow
1 reflection/scattering
2 reflection/scattering
3 reflection/scattering
4 laser process
10 self-mixing interferometry sensor module
20 light emitter
30 detector unit
40 array of light detectors
41 light detector
42 light detector
43 light detector
44 spatial offset
45 pixel
46 pixel
47 pixel
50 multilayer target
51 layer
52 layer
53 layer
60 optical element
70 optical element
100 electronic device

1. A self-mixing interferometry sensor module for multilayer target detection, comprising a light emitter, a detector unit and an array of light detectors, wherein:
the light emitter is operable to emit coherent electromagnetic radiation out of the sensor module; and undergo self-mixing interference (SMI) caused by reflections of the emitted electromagnetic radiation from layers of different depths of a multilayer target to be placed outside the sensor module;
the detector unit is operable to generate an SMI output signal indicative of the SMI of the light emitter, and
light detectors of the array are operable to generate auxiliary output signals indicative of a distribution of relative reflections of the emitted electromagnetic radiation from layers of different depths.

2. The module according to claim 1, wherein
the array of light detectors comprises a one-dimensional array or a two-dimensional array, and
neighboring light detectors are separated by a spatial offset.

3. The module according to claim 1, wherein the array of light detectors forms an image sensor.

4. The module according to claim 1, wherein the light emitter comprises:
a semiconductor laser diode,
a resonant cavity light emitting device, and/or
a vertical cavity surface emitting laser (VCSEL) diode.

5. The module according to claim 1, comprising further light emitters, wherein
the light emitters are operable to emit coherent electromagnetic radiation with a defined wavelength out of the sensor module; and undergo SMI caused by reflections of the emitted electromagnetic radiation from layers of different depths of a multilayer target to be placed outside the sensor module, and
at least two light emitters are operable to emit coherent electromagnetic radiation with different defined wavelengths.

6. The module according to claim 1, wherein the detector unit is operable to:
detect a junction voltage of a respective light emitter, and
generate the output signals as a function of said junction voltages, respectively.

7. The module according to claim 1, wherein the detector unit is operable to:
detect an optical power output of a respective light emitter, and
generate the output signals as a function of said optical power outputs, respectively.

8. The module according to claim 1, wherein the array of light detectors, the detector unit and/or at least one light emitter form an integrated semiconductor device.

9. The module according to claim 1, further comprising an electronic processing unit, which is operable to determine from the generated output signals a depth profile of the multilayer target placed outside the sensor module.

10. The module according to claim 1, wherein at least some of the light detectors are operable to generate the auxiliary output signals indicative of a distribution of relative reflections of the emitted electromagnetic radiation from layers of different depths as a function of polarization.

11. The module according to claim 1, wherein:
an optical element, e.g. a refractive, diffractive or meta-lens, is arranged in front of the light emitter to collimate or focus a diverging beam from the light emitter and/or to provide polarization control, and/or
another optical element is arranged in front of the array of light detectors, e.g. a single microlens or a microlens array) to increase a signal on the light detectors.

12. An electronic device comprising:
a self-mixing interferometry sensor module according to claim 1, and
a housing comprising the sensor module and a support surface to position the light emitter and array of light detectors at a distance from the multilayer target.

13. The device according to claim 12, further comprising a processing unit configured to determine, from an output of the module, a displacement or a movement of a sub-surface feature associated with at least one layer of the multilayer object.

14. The device according to claim 13, wherein the processing unit is operable to:
receive as an output of the module at least one SMI output signal and the auxiliary output signals, and
determine the displacement or movement of a sub-surface feature as a function of the SMI output signal and the auxiliary output signals.

15. The device according to claim 13, wherein the processing unit is further operable to combine the output of the module with a Speckle image.

16. A method of detecting a multilayer object, comprising the steps of:
placing a multilayer target outside a sensor module;
emitting coherent electromagnetic radiation out of the sensor module by means of a light emitter;
generating self-mixing interference (SMI) in the light emitter caused by reflections of the emitted electromagnetic radiation from layers of different depths of the multilayer target to be placed outside the sensor module;
generating an SMI output signal indicative of the SMI of the light emitter; and
using an array of light detectors, generating auxiliary output signals indicative of a distribution of relative reflections of the emitted electromagnetic radiation from layers of different depths.

17. A self-mixing interferometry sensor module for multilayer target detection, comprising a light emitter, a detector unit, an array of light detectors, and an electronic processing unit, wherein:
the light emitter is operable to emit coherent electromagnetic radiation out of the sensor module; and undergo self-mixing interference (SMI) caused by reflections of the emitted electromagnetic radiation from layers of different depths of a multilayer target to be placed outside the sensor module;
the detector unit is operable to generate an SMI output signal indicative of the SMI of the light emitter;
light detectors of the array are operable to generate auxiliary output signals indicative of a distribution of relative reflections of the emitted electromagnetic radiation from layers of different depths; and
the electronic processing unit is operable to determine from the generated output signals a depth profile of the multilayer target placed outside the sensor module.
